(19) United States
(12) Patent Application Publication
(10) Pub. No.: US 2024/0376038 A1
(43) Pub. Date: Nov. 14, 2024

(54) FRAGRANCE AND FLAVOR COMPOSITIONS COMPRISING HYDROXYMELONAL FORMATE ESTER

(71) Applicant: P2 SCIENCE, INC., Woodbridge, CT (US)

(72) Inventors: Patrick FOLEY, New Haven, CT (US); Yonghua YANG, New Haven, CT (US)

(21) Appl. No.: 18/692,494

(22) PCT Filed: Sep. 16, 2022
(86) PCT No.: PCT/US22/76615

§ 371 Date: Mar. 15, 2024

Related U.S. Application Data
(60) Provisional application No. 63/245,418, filed on Sep. 17, 2021.

(51) Int. Cl. C07C 69/04; C11B 9/00

(52) U.S. Cl. CPC C07C 69/04; C11B 9/00

(57) ABSTRACT

The present application relates to hydroxymelonal formate as a flavor and fragrance ingredient, compositions comprising the same, and methods of making the same.

### CROSS-REFERENCE TO RELATED APPLICATIONS

This international application claims priority to, and the benefit of, U.S. Provisional Application Ser. No. 63/245,418, filed on Sep. 17, 2021, the contents of which are hereby incorporated by reference in its entirety.

### FIELD OF THE APPLICATION

The present application relates to hydroxymelonal formate, methods of making it, and methods of using it in flavor and fragrance compositions, and products comprising such compositions.

### BACKGROUND

Scent is an important factor used to produce a sense of anticipation, quality, palatability, and security to many consumer products. Identifying effective aromas and flavors to impart in a product is an element that contributes to the success of the product, and is useful in product marketing, consumer satisfaction, and consumer retention. Sweet, resinous smells are particularly desirable fragrances and are often used in toiletries, cosmetics, household cleaners, room sprays, laundry, and fine fragrance applications, such as in perfumes and toilet waters. Similar flavors are also desirable in many dental hygiene products, orally administered medications, and food products.

Considerable work is performed by many scientists relating to identifying new substances which can be used, alone or in combinations, to impart to, or enhance, the aroma or flavor of various consumable materials, including, e.g., perfumes, tobacco, and foodstuffs. While there may be some trends in the relationship between chemical structure and flavor or fragrance—such as common use of low molecular weight aldehydes and alcohols as flavors and fragrances—the precise aroma associated with a molecule is exceedingly difficult to predict. Small changes in structure, such a lengthening or shortening a functional group by just one carbon atom, can have profound and unexpected effects on a compound's flavor or fragrance profile.

Terpenes and terpene derivatives have been used as flavors and fragrances for many years. Commonly used terpene fragrances include citronellol, citronellal, myrcenol, menthol, melonol, melonal, and certain derivatives thereof. One such derivative, for example, is hydroxycitronellal, which is reported to have a sweet-floral taste, but with a bitter aftertaste, and to have a sweet-floral aroma that strengthens over time, with widely varying top-note among different manufacturers.

Hydroxymelonal, also known as 6-hydroxy-2,6-dimethylheptanal, has been described in the literature and characterized as a useful compound in flavors and fragrances. See, e.g., U.S. Pat. No. 4,242,281 (Sprecker et al., 1980) and U.S. Pat. No. 4,311,617 (Ansari et al., 1982). Hydroxymelonal has been described as providing or enhancing sweet, green, melon-like, peach-like, tropical fruit-like, floral, seedy, and/or raspberry aromas and flavors. See Sprecker et al. It has also been described as having a powerful lily of the valley-like odor. See Ansari et al.

Hydroxymelonal formate has not, however, been described and characterized as a compound that is useful in flavor and fragrance applications. This application describes the surprising and unexpected olfactive qualities of hydroxymelonal formate, as well as mixtures of hydroxymelonal and hydroxymelonal formate, and its use as a readily accessible and cost-effective fragrance and flavor ingredient, and potential applications thereof.

### BRIEF SUMMARY

In one aspect, the application relates to the novel fragrant molecule hydroxymelonal formate, which has the following structure:

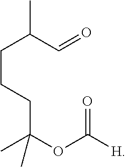

In another aspect, the application relates to fragrance and flavor compositions comprising hydroxymelonal formate, optionally comprising one or more additives, additional fragrance or flavor ingredients, or a combination of additives and fragrance or flavor ingredients. In some embodiments, such compositions comprise a mixture of hydroxymelonal and hydroxymelonal formate, e.g., in a molar ratio or mass ratio of 5:1 to 1000:1.

In another aspect, the application relates to products, such as consumer products, comprising such fragrance and flavor compositions comprising hydroxymelonal formate, e.g., products comprising fragrance and flavor compositions comprising a mixture of hydroxymelonal and hydroxymelonal formate (such as in a molar ratio or mass ratio of 5:1 to 1000:1).

In another aspect, the present disclosure provides a method of making hydroxymelonal and/or hydroxymelonal formate.

The details of one or more embodiments of the application are set forth in the accompanying description below. Unless defined otherwise, all technical and scientific terms used herein have the same meaning as commonly understood by one of ordinary skill in the art to which this application belongs. In the case of conflict, the present specification will control.

Other features and advantages of the application will be apparent from the following detailed description, examples, and claims.

### DETAILED DESCRIPTION

Hydroxymelonal formate has a unique aroma and flavor. It has thus been determined to be useful in imparting and providing desirable aromas and/or flavors to the products to which it is added. For example, hydroxymelonal formate may be used in any product where the inclusion of a pleasing fragrance or flavor is desired, including, but not limited to, perfumes, household products, laundry products, personal care products, cosmetics, dental hygiene products, orally administered medications, and food products. Hydroxymelonal formate may be employed in varying amounts depending upon the specific fragrance or flavor product application, the nature and amount of other odor-carrying ingredients present, and the desired aroma and/or flavor of the product.

Hydroxymelonal formate can be prepared by ozonolysis and reduction of dihydromyrcenol (2,6-dimethyl-2-hydroxy-7-octene) as shown below:

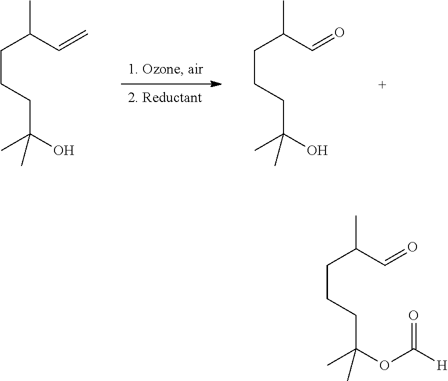

Without being bound by theory, it is believed that hydroxymelonal formate forms via an intramolecular nucleophilic attack of the hydroxy group on the intermediate cyclic ozonide, followed by reduction, for example, as follows:

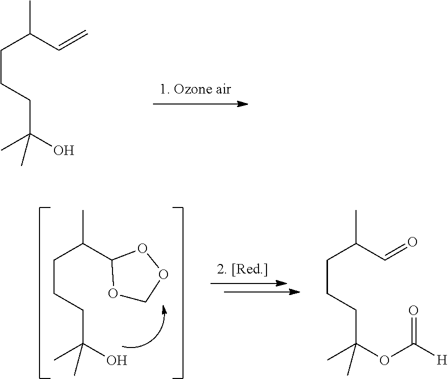

While the major product of the reaction is hydroxymelonal, the reaction conditions may be modified in order to increase or decrease the proportion of hydroxymelonal formate present in the product. In addition, as the hydroxymelonal and hydroxymelonal formate have different boiling points and different partition coefficients, various means can be used to provide a product mixture having a desired concentration of hydroxymelonal formate or ratio of hydroxymelonal-to-hydroxymelonal formate. For example, distillation or chromatography may be used to provide fractions comprising a desired mixture of hydroxymelonal and hydroxymelonal formate. Further distillation or purification may also provide hydroxymelonal formate in pure or substantially pure form, which can then be admixed with pure or substantially pure hydroxymelonal in any desired proportions to derive fragrance or flavor composition having desired properties.

In a first aspect, the present disclosure provides the novel compound hydroxymelonal formate,

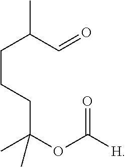

In some embodiments of the first aspect, the hydroxymelonal formate may either be in the (R) conformation, or the (S) conformation, or a mixture thereof (such as a racemic mixture). It is understood that the hydroxymelonal formate as described herein is inclusive of both the keto (aldo) tautomer and the enol tautomers thereof (the enol can be cis or trans oriented), as shown below:

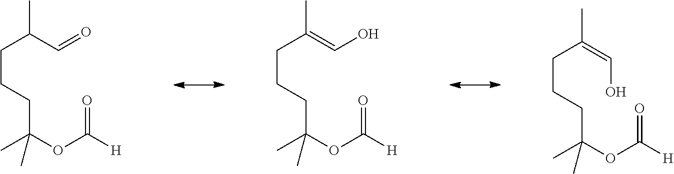

In a second aspect, the present disclosure provides a flavor composition and/or a fragrance composition (Composition 1) comprising hydroxymelonal formate. In particular embodiments, the second aspect provides:
1.1 Composition 1, wherein the composition is a fragrance composition.
1.2 Composition 1, wherein the composition is a flavor composition.
1.3 Composition 1.1 or 1.2, wherein the composition further comprises one or more solvents.
1.4 Any of Compositions 1.1-1.3, wherein the composition further comprises one or more other flavors or fragrances.
1.5 Any of Compositions 1.1-1.4, wherein the composition further comprises hydroxymelonal.
1.6 Composition 1.5, wherein the composition comprises hydroxymelonal and hydroxymelonal formate in a mass ratio of 5:1 to 1000:1, e.g., 10:1 to 1000:1, or 20:1 to 1000:1, 25:1 to 1000:1, or 50:1 to 1000:1, or 75:1 to 1000:1, or 100:1 to 1000:1, or 200:1 to 1000:1, or 250:1 to 1000:1, or 500:1 to 1000:1 or 750:1 to 1000:1, or 5:1 to 500:1, or 10:1 to 500:1, or 20:1 to 500:1, 25:1 to 500:1, or 50:1 to 500:1, or 75:1 to 500:1, or 100:1 to 500:1, or 200:1 to 500:1, or 250:1 to 500:1, or 5:1 to 250:1, or 10:1 to 250:1, or 20:1 to 250:1, 25:1 to 250:1, or 50:1 to 250:1, or 75:1 to 250:1, or 100:1 to 250:1, or 200:1 to 250:1, or 5:1 to 100:1, or 10:1 to 100:1, or 20:1 to 100:1, 25:1 to 100:1, or 50:1 to 100:1, or 75:1 to 100:1, or 5:1 to 50:1, or 10:1 to 50:1, or 20:1 to 50:1, or 25:1 to 50:1, or 5:1 to 25:1, or 10:1 to 25:1, or 15:1 to 25:1, or 20:1 to 25:1, or 5:1 to 10:1, or 5:1 to 7.5:1, or about 5:1, or about 10:1, or about 15:1, or about 25:1, or about 30:1, or about 35:1, or about 40:1, or about 45:1 or about 50:1, or about 60:1, or about 70:1 or about 75:1, or about 85:1, or about 100:1, or about 125:1, or about 150:1, or about 200:1, or about 250:1, or about 500:1, or about 750:1, or about 1000:1.
1.7 Composition 1.5 or 1.6, wherein the composition comprises hydroxymelonal and hydroxymelonal formate in a molar ratio of 5:1 to 1000:1, e.g., 10:1 to 1000:1, or 20:1 to 1000:1, 25:1 to 1000:1, or 50:1 to 1000:1, or 75:1 to 1000:1, or 100:1 to 1000:1, or 200:1 to 1000:1, or 250:1 to 1000:1, or 500:1 to 1000:1 or 750:1 to 1000:1, or 5:1 to 500:1, or 10:1 to 500:1, or 20:1 to 500:1, 25:1 to 500:1, or 50:1 to 500:1, or 75:1 to 500:1, or 100:1 to 500:1, or 200:1 to 500:1, or 250:1 to 500:1, or 5:1 to 250:1, or 10:1 to 250:1, or 20:1 to 250:1, 25:1 to 250:1, or 50:1 to 250:1, or 75:1 to 250:1, or 100:1 to 250:1, or 200:1 to 250:1, or 5:1 to 100:1, or 10:1 to 100:1, or 20:1 to 100:1, 25:1 to 100:1, or 50:1 to 100:1, or 75:1 to 100:1, or 5:1 to 50:1, or 10:1 to 50:1, or 20:1 to 50:1, or 25:1 to 50:1, or 5:1 to 25:1, or 10:1 to 25:1, or 15:1 to 25:1, or 20:1 to 25:1, or 5:1 to 10:1, or 5:1 to 7.5:1, or about 5:1, or about 10:1, or about 15:1, or about 25:1, or about 30:1, or about 35:1, or about 40:1, or about 45:1 or about 50:1, or about 60:1, or about 70:1 or about 75:1, or about 85:1, or about 100:1, or about 125:1, or about 150:1, or about 200:1, or about 250:1, or about 500:1, or about 750:1, or about 1000:1.
1.8 Any of compositions 1.5-1.7, wherein the composition consists of hydroxymelonal and hydroxymelonal formate.
1.9 Composition 1.8, wherein the composition consists of 0.1 to 20% by weight of hydroxymelonal formate, wherein the balance of the mass is hydroxymelonal, e.g., wherein the composition consists of 0.1 to 15%, or 0.1 to 10%, or 0.1 to 7.5%, or 0.1 to 5%, or 0.1 to 4%, or 0.1 to 3%, or 0.1 to 2%, or 0.1 to 1%, or 0.1 to 0.8%, or 0.1 to 0.6%, or 0.1 to 0.5%, or 0.1 to 0.4%, or 0.1 to 0.3%, or 0.1 to 0.2%, or 0.1 to 0.15%, or 0.5% to 15%, or 0.5% to 10%, or 0.5 to 7.5%, or 0.5 to 5%, or 0.5 to 4%, or 0.5 to 3%, or 0.5 to 2%, or 0.5 to 1%, or 0.5 to 0.8%, or 0.5 to 0.6%, or 1 to 15%, or 1 to 10%, or 1 to 7.5%, or 1 to 5%, or 1 to 4%, or 1 to 3%, or 1 to 2%, or 1 to 1.5%, or 2 to 15%, or 2 to 10%, or 2 to 7.5%, or 2 to 5%, or 2 to 4%, or 2 to 3%, or 2 to 2.5%, or 3 to 15%, or 3 to 10%, or 3 to 7.5%, or 3 to 5%, or 5 to 15%, or 5 to 10%, or 5 to 7.5%, or 7.5% to 15%, or 7.5% to 10%, or 10 to 15%, by weight of hydroxymelonal formate, and the balance of the mass is hydroxymelonal.
1.10 Any of compositions 1.5-1.7, wherein the composition comprises or consists of hydroxymelonal, hydroxymelonal formate, and one or more solvents.
1.11 Composition 1.10, wherein the composition comprises or consists of hydroxymelonal, hydroxymelonal formate, and a net amount of solvent of 1 to 99% by weight of the composition, e.g., 10 to 99%, or 15 to 99%, or 20 to 99%, or 25 to 99%, or 30 to 99%, or 35 to 99%, or 40 to 99%, or 45 to 99%, or 50 to 99%, or 60 to 99%, or 70 to 99%, or 80 to 99%, or 90 to 99%, or 95 to 99%, or 98 to 99%, the balance being hydroxymelonal and hydroxymelonal formate.
1.12 Any of compositions 1.5-1.7, wherein the composition comprises or consists of hydroxymelonal, hydroxymelonal formate, one or more solvents, and one or more other flavors or fragrances.
1.13 Composition 1.12, wherein the composition comprises or consists of hydroxymelonal, hydroxymelonal formate, one or more solvents in a net amount of 1 to 99% by weight of the composition, e.g., 10 to 99%, or 15 to 99%, or 20 to 99%, or 25 to 99%, or 30 to 99%, or 35 to 99%, or 40 to 99%, or 45 to 99%, or 50 to 99%, or 60 to 99%, or 70 to 99%, or 80 to 99%, or 90 to 99%, or 95 to 99%, or 98 to 99%, and one or more other flavors or fragrances in a net amount of 0.1 to 15% by weight of the composition, e.g., 0.1 to 10%, or 0.1 to 7.5%, or 0.1 to 5%, or 0.1 to 4%, or 0.1 to 3%, or 0.1 to 2%, or 0.1 to 1%, or 0.1 to 0.5%, the balance being hydroxymelonal and hydroxymelonal formate.
1.14 Any of compositions 1.3 to 1.13, wherein the one or more solvents are selected from water, methanol, ethanol, propanol, isopropanol, dimethyl ether, diethyl ether, diisopropyl ether, methyl tert-butyl ether, ethylene glycol, propylene glycol, glycerin, triethyl citrate, triacetin, triglycerides, liquid waxes, propylene glycol derivatives (e.g., polypropylene glycols or ethylene oxide/propylene oxide copolymers), ethylene glycol derivatives (e.g., polyethylene glycols or ethylene oxide/propylene oxide copolymers), other alcohols or ethers, or any combination thereof.
1.15 Any of composition 1.1 to 1.14, wherein the composition is a liquid.
1.16 Any of compositions 1.1 to 1.14, wherein the composition is a soft or waxy solid.
1.17 Any of compositions 1.1-1.16, wherein the composition further comprises one or more of a polymer, gelling agent, powdery substrate, surfactant, emollient, plasticizer, wetting agent, swelling agent, or active agent (e.g., an oral care active or a medicinal active agent), or any other additives as described herein.

As used herein, the term “fragrance composition” means a mixture of fragrance ingredients, including auxiliary substances if desired, dissolved in a suitable solvent or mixed with a powdery substrate used to provide a desired odor to a product. Examples of products having fragrance compositions include, but are not limited to, perfumes, soaps, insect repellants and insecticides, detergents, household cleaning agents, air fresheners, room sprays, pomanders, candles, cosmetics, toilet waters, pre- and aftershave lotions, talcum powders, hair-care products, body deodorants, anti-perspirants, and pet litter.

As used herein, the term “flavor composition” means a mixture of flavor ingredients, including auxiliary substances if desired, dissolved in a suitable solvent or mixed with a powdery substrate used to provide a desired flavor to a product. Examples of products having flavor compositions include, but are not limited to, dental hygiene products such as mouth wash, toothpaste, floss, and breath fresheners, orally administered medications including liquids, tablets or capsules, and food products.

Fragrance and flavor ingredients and mixtures of fragrance and flavor ingredients that may be used in combination with the disclosed compound for the manufacture of fragrance and flavor compositions include, but are not limited to, natural products including extracts, animal products and essential oils, absolutes, resinoids, resins, and concretes, and synthetic fragrance materials which include, but are not limited to, alcohols, aldehydes, ketones, ethers, acids, esters, acetals, phenols, ethers, lactones, furansketals, nitriles, acids, and hydrocarbons, including both saturated and unsaturated compounds and aliphatic carbocyclic and heterocyclic compounds, and animal products. As used herein, the terms “fragrance ingredient” and “flavor ingredient” refer to ingredients other than hydroxymelonal and hydroxymelonal formate which are used to impart a flavor or a fragrance to a composition or product.

Examples of esters which may be used as fragrance ingredients or flavor ingredients in the compositions and products of the present disclosure include, but are not limited to, acrylic acid esters (methyl, ethyl, etc.), acetoacetic acid esters (methyl, ethyl, etc.), anisic acid esters (methyl, ethyl, etc.), benzoic acid esters (allyl, isoamyl, ethyl, geranyl, linalyl, phenylethyl, hexyl, cis-3-hexenyl, benzyl, methyl, etc.), anthranilic acid esters (cinnamyl, cis-3-hexenyl, methyl, ethyl, linalyl, isobutyl, etc.), N-methylanthranilic acid esters (methyl, ethyl, etc.), isovaleric acid esters (amyl, allyl, isoamyl, isobutyl, isopropyl, ethyl, octyl, geranyl, cyclohexyl, citronellyl, terpenyl, linalyl, cinnamyl, phenylethyl, butyl, propyl, hexyl, benzyl, methyl, rhodinyl, etc.), isobutyric acid esters (isoamyl, geranyl, citronellyl, terpenyl, cinnamyl, octyl, nellyl, phenylethyl, phenylpropyl, phenoxyethyl, butyl, propyl, isopropyl, hexyl, benzyl, methyl, ethyl, linalyl, rhodinyl, etc.), undecylenic acid esters (allyl, isoamyl, butyl, ethyl, methyl, etc.), octanoic acid esters (allyl, isoamyl, ethyl, octyl, hexyl, butyl, methyl, linalyl, etc.), octenoic acid esters (methyl, ethyl, etc.), octynecarboxylic acid esters (methyl, ethyl, etc.), caproic acid esters (allyl, amyl, isoamyl, methyl, ethyl, isobutyl, propyl, hexyl, cis-3-hexenyl, trans-2-hexenyl, linalyl, geranyl, cyclohexyl, etc.), hexenoic acid esters (methyl, ethyl, etc.), valeric acid esters (amyl, isopropyl, isobutyl, ethyl, cis-3-hexenyl, trans-2-hexenyl, cinnamyl, phenylethyl, methyl, etc.), formic acid esters (anisyl, isoamyl, isopropyl, ethyl, octyl, geranyl, citronellyl, cinnamyl, cyclohexyl, terpenyl, phenylethyl, butyl, propyl, hexyl, cis-3-hexenyl, benzyl, linalyl, rhodinyl, etc.), crotonic acid esters (isobutyl, ethyl, cyclohexyl, etc.), cinnamic acid esters (allyl, ethyl, methyl, isopropyl, propyl, 3-phenylpropyl, benzyl, cyclohexyl, methyl, etc.), succinic acid esters (monomenthyl, diethyl, dimethyl, etc.), acetic acid esters (anisyl, amyl, α-amylcinnamyl, isoamyl, isobutyl, isopropyl, isobornyl, isoeugenyl, eugenyl, 2-ethylbutyl, ethyl, 3-octyl, p-cresyl, o-cresyl, geranyl, α- or β-santalyl, cyclohexyl, cycloneryl, dihydrocuminyl, dimethyl benzyl carbinyl, cinnamyl, styralyl, decyl, dodecyl, terpenyl, guainyl, neryl, nonyl, phenyl ethyl, phenylpropyl, butyl, furfuryl, propyl, hexyl, cis-3-hexenyl, trans-2-hexenyl, cis-3-nonenyl, cis-6-noneyl, cis-3-cis-6-nonadienyl, 3-methyl-2-butenyl, heptyl, benzyl, bornyl, myrcenyl, dihydromyrcenyl, myrtenyl, methyl, 2-methylbutyl, menthyl, linalyl, rhodinyl, etc.), salicylic acid esters (allyl, isoamyl, phenyl, phenylethyl, benzyl, ethyl, methyl, etc.), cyclohexylalkanoic acid esters (ethyl cyclohexylacetate, allyl cyclohexylpropionate, allyl cyclohexylbutyrate, allyl cyclohexylhexanoate, allyl cyclohexyldecanoate, allyl cyclohexylvalerate, etc.), stearic acid esters (ethyl, propyl, butyl, etc.), sebacic acid esters (diethyl, dimethyl, etc.), decanoic acid esters (isoamyl, ethyl, butyl, methyl, etc.), dodecanoic acid esters (isoamyl, ethyl, butyl, etc.), lactic acid esters (isoamyl, ethyl, butyl, etc.), nonanoic acid esters (ethyl, phenylethyl, methyl, etc.), nonenoic acid esters (allyl, ethyl, methyl, etc.), hydroxyhexanoic acid esters (ethyl, methyl, etc.), phenylacetic acid esters (isoamyl, isobutyl, ethyl, geranyl, citronellyl, cis-3-hexenyl, methyl, etc.), phenoxyacetic acid esters (allyl, ethyl, methyl, etc.), furancarboxylic acid esters (ethyl furancarboxylate, methyl furancarboxylate, hexyl furancarboxylate, isobutyl furaneopentyl glycol diacetateropionate, etc.), propionic acid esters (anisyl, allyl, ethyl, amyl, isoamyl, propyl, butyl, isobutyl, isopropyl, benzyl, geranyl, cyclohexyl, citronellyl, cinnamyl, tetrahydrofurfuryl, tricyclodecenyl, heptyl, bornyl, methyl, menthyl, linallyl, terpenyl, α-methylpropionyl, β-methylpropionyl, etc.), heptanoic acid esters (allyl, ethyl, octyl, propyl, methyl, etc.), heptinecarboxylic acid esters (allyl, ethyl, propyl, methyl, etc.), myristic acid esters (isopropyl, ethyl, methyl, etc.), phenylglycidic acid esters (ethyl phenylglycidate, ethyl 3-methylphenylglycidate, ethyl β-methyl-β-phenylglycidate, etc.), 2-methylbutyric acid esters (methyl, ethyl, octyl, phenyl ethyl, butyl, hexyl, benzyl, etc.), 3-methylbutyric acid esters (methyl, ethyl, etc.), butyric acid esters (anisyl, amyl, allyl, isoamyl, methyl, ethyl, propyl, octyl, guainyl, linallyl, geranyl, cyclohexyl, citronellyl, cinnamyl, nellyl, terpenyl, phenylpropyl, β-phenylethyl, butyl, hexyl, cis-3-hexenyl, trans-2-hexenyl, benzyl, rhodinyl, etc.), and hydroxybutyric acid esters (methyl, ethyl, menthyl or the like of 3-hydroxybutyric acid esters).

Examples of alcohols which may be used as fragrance ingredients or flavor ingredients, or as solvents, in the compositions and products of the present disclosure include, but are not limited to, aliphatic alcohols (isoamyl alcohol, 2-ethylhexanol, 1-octanol, 3-octanol, 1-octene-3-ol, 1-decanol, 1-dodecanol, 2,6-nonadienol, nonanol, 2-nonanol, cis-6-nonenol, trans-2, cis-6-nonadienol, cis-3, cis-6-nonadienol, butanol, hexanol, cis-3-hexenol, trans-2-hexenol, 1-undecanol, heptanol, 2-heptanol, 3-methyl-1-pentanol, etc.); terpene alcohols (borneol, isoborneol, carveol, geraniol, α- or β-santalol, citronellol, 4-thujanol, terpineol, 4-terpineol, nerol, myrcenol, myrtenol, dihydromyrcenol, tetrahydromyrcenol, nerolidol, hydroxycitronellol, farnesol, perilla alcohol, rhodinol, linalool, etc.); and aromatic alcohols (anisic alcohol, α-amylcinnamic alcohol, isopropylbenzylcarbinol, carvacrol, cumin alcohol, dimethylbenzylcarbinol, cinnamic alcohol, phenyl allyl alcohol, phenylethylcarbinol, β-phenylethyl alcohol, 3-phenylpropyl alcohol, benzyl alcohol, etc.).

Examples of aldehydes which may be used as fragrance ingredients or flavor ingredients in the compositions and products of the present disclosure include, but are not limited to, aliphatic aldehydes (acetaldehyde, octanal, nonanal, decanal, undecanal, 2,6-dimethyl-5-heptanal, 3,5,5-trimethylhexanal, cis-3, cis-6-nonadienal, trans-2, cis-6-nonadienal, valeraldehyde, propanal, isopropanal, hexanal, trans-2-hexenal, cis-3-hexenal, 2-pentenal, dodecanal, tetradecanal, trans-4-decenal, trans-2-tridecenal, trans-2-dodecenal, trans-2-undecenal, 2,4-hexadienal, cis-6-nonenal, trans-2-nonenal, 2-methylbutanal, etc.); aromatic aldehydes (anisic aldehyde, α-amylcinnamic aldehyde, α-methylcinnamic aldehyde, cyclamen aldehyde, p-isopropylphenylacetaldehyde, ethylvanillin, cumin aldehyde, salicylaldehyde, cinnamic aldehyde, o-, m- or p-tolylaldehyde, vanillin, piperonal, phenylacetaldehyde, heliotropin, benzaldehyde, 4-methyl-2-pheny-2-pentenal, p-methoxycinnamic aldehyde, p-methoxybenzaldehyde, etc.); and terpene aldehydes (geranial, citral, citronellal, α-sinensal, β-sinensal, perillaldehyde, hydroxycitronellal, tetrahydrocitral, myrtenal, cyclocitral, isocyclocitral, citronellyloxyacetaldehyde, neral, α-methylenecitronellal, myracaldehyde, vernaldehyde, safranal, etc.).

Examples of ketones which may be used as fragrance ingredients or flavor ingredients in the compositions and products of the present disclosure include, but are not limited to, cyclic ketones (1-acetyl-3,3-dimethyl-1-cyclohexene, cis-jasmone, α-, β- or γ-irone, ethyl maltol, cyclotene, dihydronootkatone, 3,4-dimethyl-1,2-cyclopentadione, sotolon, α-, β-, γ- or δ-damascone, α-, β- or γ-damascenone, nootkatone, 2-sec-butylcyclohexanone, maltol, α-, β- or γ-ionone, α-, β- or γ-methylionone, α-, β- or γ-isomethylionone, furaneol, camphor, etc.); aromatic ketones (acetonaphthone, acetophenone, anisylideneacetone, raspberry ketone, p-methyl acetophenone, anisylacetone, p-methoxy acetophenone, etc.); and chain ketones (diacetyl, 2-nonanone, diacetyl, 2-heptanone, 2,3-heptanedione, 2-pentanone, methyl amyl ketone, methyl nonyl ketone, β-methyl naphthyl ketone, methyl heptanone, 3-heptanone, 4-heptanone, 3-octanone, 2,3-hexanedione, 2-undecanone, dimethyloctenone, 6-methyl-5-hepten-2-one, etc.).

Examples of acetals which may be used as fragrance ingredients or flavor ingredients in the compositions and products of the present disclosure include, but are not limited to, acetaldehyde diethyl acetal, acetaldehyde diamyl acetal, acetaldehyde dihexyl acetal, acetaldehyde propylene glycol acetal, acetaldehyde ethyl cis-3-hexenyl acetal, benzaldehyde glycerin acetal, benzaldehyde propylene glycol acetal, citral dimethyl acetal, citral diethyl acetal, citral propylene glycol acetal, citral ethylene glycol acetal, phenylacetaldehyde dimethyl acetal, citronellyl methyl acetal, acetaldehyde phenylethylpropyl acetal, hexanal dimethyl acetal, hexanal dihexyl acetal, hexanal propylene glycol acetal, trans-2-hexenal diethyl acetal, trans-2-hexenal propylene glycol acetal, cis-3-hexenal diethyl acetal, heptanal diethyl acetal, heptanal ethylene glycol acetal, octanal dimethyl acetal, nonanal dimethyl acetal, decanal dimethyl acetal, decanal diethyl acetal, 2-methylundecanal dimethyl acetal, citronellal dimethyl acetal, Ambersage (manufactured by Givaudan), ethyl acetoacetate ethylene glycol acetal, and 2-phenylpropanal dimethyl acetal.

Examples of phenols which may be used as fragrance ingredients or flavor ingredients in the compositions and products of the present disclosure include, but are not limited to, eugenol, isoeugenol, 2-methoxy-4-vinylphenol, thymol, carvacrol, guaiacol, and chavicol, and vanillin.

Examples of ethers and epoxides which may be used as fragrance ingredients or flavor ingredients in the compositions and products of the present disclosure, but are not limited to, anethole, 1,4-cineole, dibenzyl ether, linalool oxide, limonene oxide, nerol oxide, rose oxide, methyl isoeugenol, methyl chavicol, isoamyl phenyl ethyl ether, β-naphtyl methyl ether, phenyl propyl ether, p-cresyl methyl ether, vanillyl butyl ether, α-terpinyl methyl ether, citronellyl ethyl ether, geranyl ethyl ether, rosefuran, theaspirane, decylmethyl ether, and methylphenyl methyl ether.

Examples of lactones which may be used as fragrance ingredients or flavor ingredients in the compositions and products of the present disclosure include, but are not limited to, γ- or δ-decalactone, γ-heptalactone, γ-nonalactone, γ- or δ-hexylactone, γ- or δ-octalactone, γ- or δ-undecalactone, δ-dodecalactone, δ-2-decenolactone, methyl lactone, 5-hydroxy-8-undecenoic acid δ-lactone, jasmine lactone, menthalactone, dihydrocoumarin, octahydrocoumarin, and δ-methylcoumarin.

Examples of furans which may be used as fragrance ingredients or flavor ingredients in the compositions and products of the present disclosure include, but are not limited to, furan, 2-methylfuran, 3-methylfuran, 2-ethylfuran, 2,5-diethyltetrahydrofuran, 3-hydroxy-2-methyltetrahydrofuran, 2-(methoxymethyl)furan, 2,3-dihydrofuran, furfural, 5-methylfurfural, 3-(2-furyl)-2-methyl-2-propenal, 5-(hydroxymethyl)furfural, 2,5-dimethyl-4-hydroxy-3(2H)-furanone (furaneol), 4,5-dim ethyl-3-hydroxy-2(5H)-furanone (sotolon), 2-ethyl-4-hydroxy-5-methyl-3(2H)-furanone (homofuraneol), 5-ethyl-3-hydroxy-4-methyl-2(5H)-furanone (homosotolon), 3-methyl-1,2-cyclopentanedione (cyclotene), 2(5H)-furanone, 4-methyl-2(5H)-furanone, 5-methyl-2(5H)-furanone, 2-methyl-3(2H)-furanone, 5-methyl-3(2H)-furanone, 2-acetylfuranone, 2-acetyl-5-methylfuran, furfuryl alcohol, methyl 2-furancarboxylate, ethyl 2-furancarboxylate, and furfuryl acetate.

Examples of hydrocarbons which may be used which may be used as fragrance ingredients or flavor ingredients in the compositions and products of the present disclosure include, but are not limited to, α- or β-bisabolene, β-caryophyllene, p-cymene, terpinene, terpinolene, cadinene, cedrene, longifolene, farnesene, limonene, ocimene, myrcene, α- or β-pinene, 1,3,5-undecatriene and valencene.

Examples of acids that may be used which may be used as fragrance ingredients or flavor ingredients in the compositions and products of the present disclosure include, but are not limited to, geranic acid, dodecanoic acid, myristic acid, stearic acid, lactic acid, phenylacetic acid, pyruvic acid, trans-2-methyl-2-pentenoic acid, 2-methyl-cis-3-pentenoic acid, 2-methyl-4-pentenoic acid, and cyclohexanecarboxylic acid.

The fragrance and flavor compositions of the application may comprise as additional fragrance or flavor ingredients one or more natural extracts or oils including, but not limited to, anise, orange, lemon, lime, mandarin, petitgrain, bergamot, lemon balm, grapefruit, elemi, olibanum, lemongrass, neroli, marjoram, angelica root, star anise, basil, bay, calamus, chamomile, caraway, cardamom, cassia, cinnamon, pepper, perilla, cypress, oregano, cascarilla, ginger, parsley, pine needle, sage, hyssop, tea tree, mustard, horseradish, clary sage, clove, cognac, coriander, estragon, eucalyptus, fennel, guaiac wood, dill, cajuput, wormseed, pimento, juniper, fenugreek, garlic, laurel, mace, myrrh, nutmeg, spruce, geranium, citronella, lavender, lavandin, palmarosa, rose, rosemary, sandalwood, oakmoss, cedarwood, vetiver, linaloe, bois de rose, patchouli, labdanum, cumin, thyme, ylang ylang, birch, capsicum, celery, tolu balsam, genet, immortelle, benzoin, jasmine, cassie, tuberose, reseda, marigold, mimosa, opoponax, orris, vanilla and licorice. Each of these natural extracts or oils comprises a complex mixture of chemical compounds, which may include those compounds described above. Additional fragrance ingredients may be isolated from natural products, for example, geraniol and citronellal may be isolated from citronella oil, citral may be isolated from lemon-grass oil, eugenol may be isolated from clove oil, and linalool may be isolated from rosewood oil. Animal products used in fragrance compositions include, but are not limited to, musk, ambergris, civet and castoreum. The natural ingredients described herein may also be produced synthetically, and may include the compounds disclosed herein, and be used as fragrance and/or flavor ingredients in the fragrance and flavor compositions of the present application.

Examples of fragrance ingredients used in perfumes, air fresheners, laundry detergents, pet litters, cleaning products, liquid and bar soaps, shampoos and conditioners, cosmetics, deodorants, and personal hygiene products include, but are not limited to: hexyl cinnamic aldehyde; amyl cinnamic aldehyde; amyl salicylate; hexyl salicylate; terpineol; 3,7-dimethyl-cis-2,6-octadien-1-ol; 2,6-dimethyl-2-octanol; 2,6-dimethyl-7-octen-2-ol; 3,7-dimethyl-3-octanol; 3,7-dimethyl-trans-2,6-octadien-1-ol; 3,7-dimethyl-δ-octen-1-ol; 3,7-dimethyl-1-octanol; 2-methyl-3-(para-tert-butylphenyl)-propionaldehyde; 4-(4-hydroxy-4-methylpentyl)-3-cyclohexene-1-carboxaldehyde; tricyclodecenyl propionate; tricyclodecenyl acetate; anisaldehyde; 2-methyl-2-(para-iso-propylphenyl)-propionaldehyde; ethyl-3-methyl-3-phenyl glycidate; 4-(para-hydroxyphenyl)-butan-2-one; 1-(2,6,6-trimethyl-2-cyclohexen-1-yl)-2-buten-1-one; para-methoxyacetophenone; para-methoxy-alpha-phenylpropene; methyl-2-n-hexyl-3-oxo-cyclopentane carboxylate; undecalactone gamma, geraniol; geranyl acetate; linalool; linalyl acetate; tetrahydrolinalool; citronellol; citronellyl acetate; dihydromyrcenol; dihydromyrcenyl acetate; tetrahydromyrcenol; terpinyl acetate; nopol; nopyl acetate; 2-phenylethanol; 2-phenylethyl acetate; benzyl alcohol; benzyl acetate; benzyl salicylate; benzyl benzoate; styrallyl acetate; dimethylbenzylcarbinol; trichloromethylphenylcarbinyl methylphenylcarbinyl acetate; isononyl acetate; vetiveryl acetate; vetiverol; 2-methyl-3-(p-tert-butylphenyl)-propanal; 2-methyl-3-(p-isopropylphenyl)-propanal; 3-(p-tert-butylphenyl)-propanal; 4-(4-methyl-3-pentenyl)-3-cyclohexenecarbaldehyde; 4-acetoxy-3-pentyltetrahydropyran; methyl dihydrojasmonate; 2-n-heptylcyclopentanone; 3-methyl-2-pentyl-cyclopentanone; n-decanal; n-dodecanal; 9-decenol-1; phenoxyethyl isobutyrate; phenylacetaldehyde dimethylacetal; phenylacetaldehyde diethylacetal; geranonitrile; citronellonitrile; cedryl acetal; 3-isocamphylcyclohexanol; cedryl methylether; isolongifolanone; aubepine nitrile; aubepine; heliotropine; eugenol; vanillin; diphenyl oxide; hydroxycitronellal ionones; methyl ionones; isomethyl ionomes; irones; cis-3-hexenol and esters thereof; indane musk fragrances; tetralin musk fragrances; isochroman musk fragrances; macrocyclic ketones; macrolactone musk fragrances; and ethylene brassylate.

The fragrance and flavor ingredients in a given product's fragrance or flavor composition are selected based on the intended use of the product and the product's desired aroma. For example, flavor ingredients used in toothpaste, mouth wash, and dental hygiene products may be selected to impart “freshness” and include, but are not limited to, spearmint oil, peppermint oil, star anise oil, lemon oil, and menthol.

Flavor compositions may be used to mask the unpleasant taste of orally administered medications. For example, if a medication is salty, a flavor composition that has cinnamon, raspberry, orange, maple, butterscotch, or glycyrrhiza (licorice) flavor may be used to mask the taste. If a medication is overly sweet, a flavor composition that has a berry, vanilla, or acacia flavor may render the medication more palatable. In the case of bitter tasting medications, flavor compositions that have cocoa, chocolate-mint, wild cherry, walnut, glycyrrhiza (licorice), and eriodictyon flavors might be used, whereas sour medications may be improved by flavor compositions that have fruity, citrus, or cherry flavors. These flavors may be provided by the natural or synthetic flavor ingredients discussed herein.

Examples of flavor ingredients used in flavor compositions for food products also include, but are not limited to, glucosyl steviol glycosides, isomenthols, carbonothoic acids, cassyrane, 1,5-octadien-3-ol, 2-mercaptoheptan-4-ol, 4 3-(methylthio)decanal, (4Z,7Z)-trideca-4,7-dienal, Persicaria odorata oil, Amacha leaves extract, glutamyl-2-aminobutyric acid, glutamyl-2-aminobutyric acid, glutamyl-norvalyl-glycine, glutamyl-norvaline, N1-(2,3-Dimethoxybenzyl)-N2-(2-(pyridin-2-yl)ethyl) oxalamide, 1-(2-hydroxy-4-methylcyclohexyl)ethanone, Mexican lime oil, Persian lime oil, δ-methoxy-2,6-dimethylheptanal, 3,5-undecadien-2-one, 2,5-undecadien-1-ol, triethylthialdine. 4-methylpentyl 4-methylvalerate, (R)—N-(1-methoxy-4-methylpentan-2-yl)-3,4-dimethylbenzamide, 2 N-acetyl glutamate, 1,3-propanediol, Szechuan pepper extract, Tasmannia lanceolata extract, Mentha longifolia oil, mangosteen distillate, ethyl 3-(2-hydroxyphenyl)propanoate, 1-cyclopropanemethyl-4-methoxybenzene, prenyl thioisobutyrate, prenyl thioisovalerate, matairesinol, stevioside, 1-(2,4-dihydroxyphenyl)-3-(3-hydroxy-4-methoxyphenyl)propan-1-one, ethyl 5-formyloxydecanoate, 3-[3-(2-isopropyl-5-methyl-cyclohexyl)ureido]butyric acid ethyl ester, 2-Isopropyl-4-methyl-3-thiazoline, 2,6,10-trimethyl-9-undecenal, 5-mercapto-5-methyl-3-hexanone, Meyer lemon oil, teviol glycoside extract, Stevia rebaudiana, rebaudioside A 60%, rubescenamine, 4-amino-5-(3-(isopropylamino)-2,2-dimethyl-3-oxopropoxy)-2-methylquinoline-3-carboxylic acid, 3-methyl-5-(2,2,3-trimethylcyclopent-3-en-1-yl)pent-4-en-2-ol, (1-Methyl-2-(1,2,2-trimethylbicyclo[3.1.0]hex-3-ylmethyl)cyclopropyl)methanol, erospicata oil, and curly mint oil. See L. J. Marnett et al., GRAS Flavoring Substances 26, Food Technology, 44-45 (2013).

Preferred solvents for use in the compositions and products of the present disclosure include, but are not limited to, triethyl citrate, triacetin, glycerol, ethanol, water, triglycerides, liquid waxes, propylene glycol derivatives, and ethylene glycol derivatives.

The amount of a given fragrance or flavor ingredient in a fragrance or flavor composition cannot be categorically described because it varies depending on the type product being scented or flavored, the intended use of the product, and the desired aroma and/or taste of the product. The amount of a fragrance or flavor ingredient in a fragrance or flavor composition is usually in the range of from about 1% to about 99% by mass of the fragrance composition. When the amount of the ingredient is too small, a sufficient strength of the scent or flavor may not be obtained. Further, when the amount of the ingredient is too large, a larger amount of the agent(s) needed to solubilize the ingredient may be needed, which may in turn reduce the desired aromatic or flavor properties of the end product by inhibiting volatilization or other mechanisms by which the flavor or fragrance is dispersed when the product is used or consumed. The amount of each of the fragrance and flavor ingredients in a given fragrance or flavor composition must therefore be selected based upon the aromatic and/or flavor characteristics of the selected ingredient, the overall composition of the product, and the intended aromatic and/or flavor effect.

Additives may be used in the flavor and fragrance compositions of the present disclosure. Additives that may be used include, but are not limited to, solvents, surfactants, pH adjusters, buffers, thickening agents, desiccants, emulsifiers, foaming agents, stabilizers, antioxidants, and disintegrating agents. Other fragrance and flavor composition additives will be selected in accordance with the intended use of the composition.

Solvents, for example water-soluble organic solvents, which may be used in the flavor and fragrance compositions of the present disclosure include, but are not limited to, ethanol, propanol, isopropanol, butanol, 3-methoxy-3-methyl-1-butanol, benzyl alcohol, ethyl carbitol (diethylene glycol monoethyl ether), ethylene glycol, propylene glycol, dipropylene glycol, butylene glycol, hexylene glycol, glycerin, ethylene glycol monomethyl ether, propylene glycol monomethyl ether, propylene glycol monoethyl ether, and dipropylene glycol monomethyl ether. These water-soluble solvents may be used solely or in combination. The content of the water-soluble organic solvent in the compositions of the application may be determined according to the desired composition properties, and is usually from about 1% to about 99% by mass.

Oil-soluble organic solvents which may be used with the flavor and fragrance compositions of the application include, but are not limited to, isoparaffin, paraffin, limonene, pinene, triethyl citrate, benzyl benzoate, isopropyl myristate, triacetin, and silicon.

Preferred solvents include, but are not limited to, triethyl citrate, triacetin, glycerol, ethanol, water, triglycerides, liquid waxes, propylene glycol derivatives, and ethylene glycol derivatives.

In some embodiments, the flavor and fragrance compositions and products of the present disclosure may further comprise other substances, including, but not limited to, sequestering agents, preservatives, antioxidants, deodorizers, sterilization agents, ultraviolet absorbers, pH adjusters, insecticidal components, components for protection from insects, insect repellents, colorants, excipients, and buffers. The substances used in, or in addition to, the fragrance and flavor compositions of the present application may be determined by the product in which the composition is included. When the substance is used in a flavor or fragrance composition, it may be an additive. When the substance is used alongside a flavor or fragrance composition, it may be considered as part of a product composition that comprises a fragrance or flavor composition.

Excipients that may be used in the fragrance and flavoring compositions and products of the present disclosure may vary depending on the use of the intended product and its overall composition. In some instances, the excipient may be included in the fragrance or flavor composition or may, alternatively, be independent of the composition. Excipients used in or with flavoring compositions of an orally administered medication may include, but are not limited to, tablet coatings, such as a cellulose ether hydroxypropyl methylcellulose, synthetic polymer, shellac, corn protein zein or other polysaccharides, and gelatin. In pet litter, a solid excipient comprised of cellulosic or chlorophyll-containing agents or other materials may be used. In contrast, cosmetic excipients may include, but are not limited to, Carbopol 940 ETD, triethanolamine, purified water, glycerin, imidazolidinyl urea, EDTA, 1polyvinyl alcohol, methyl parabens phenoxyethanol 0, ethyl alcohol 1, peg 7 glyceryl cocoate, peg 6 triglyceryl caproic glycerides, acemulogar LAM V, isopropyl myristate, tegosoft CT, xanthan gum, sepicide CL, polyquaternium 7, and Vaseline oils. Additional suitable excipients for use with or in a flavor and/or fragrance composition for a given product will be readily selected by those having ordinary skill in the art.

Buffers that may be used with the fragrance and flavoring compositions of the present application may vary depending on the use of the intended product and its overall composition. In some instances, the buffer may be included in the fragrance or flavor composition or may, alternatively, be independent of the composition. Examples of buffers that may be used in or with the fragrance and flavor compositions of the application include, but are not limited to, citrates, acetates, and phosphates. For example, trisodium citrate may be used as a flavor or as a preservative, and is known to impart tartness to a flavor, but also acts as a buffer. Trisodium citrate is an ingredient in a variety of sodas and other beverages, as well as drink mixes and bratwurst. In cosmetic products, disodium hydrogen phosphate, potassium dihydrogen phosphate, disodium hydrogen phosphate and, and citric acid may be used to buffer the pH of the product. In toothpaste, calcium carbonate and/or dicalcium phosphate may be used as pH buffers. Additional suitable buffers for use with or in a flavor and/or fragrance composition for a given product will be readily selected by those having ordinary skill in the art.

In a fourth aspect, the present disclosure provides a product which comprises Composition 1 or any of 1.1 to 1.17. In some embodiments, the product may be selected from the following: personal care products (e.g., a soap, skin cream or lotion, balm, shampoo, body wash, shower gel, hydrating cream, deodorant, antiperspirant, after-shave lotion, cologne, perfume, or other hair care or skin care product), sunscreens, insect repellants and insecticides, detergents, household cleaning agents (e.g., a surface cleaner, a metal cleaner, a wood cleaner, a glass cleaner, a body cleaner such as a soap, a dish-washing detergent, or a laundry detergent), air fresheners, room sprays, pomanders, candles, cosmetics (e.g., perfumes, colognes, nail polish, eye liner, mascara, lipstick, foundation, concealer, blush, bronzer, eye shadow, lip liner, lip balm), toilet waters, talcum powders, and pet litter.

In fourth aspect, the present disclosure provides a method (Method 1) for the preparation of hydroxymelonal formate and/or hydroxymelonal. In further embodiments of the fourth aspect, the present disclosure provides:
1.1. Method 1, wherein the method comprises the step of reacting dihydromyrcenol with ozone for a sufficient amount of time to form an ozonide intermediate and/or to form the hydroxymelonal formate product by intramolecular reaction.
1.2. Method 1.1, wherein the ozone is supplied as a mixture with a carrier gas, e.g., air, oxygen, nitrogen, or an oxygen/nitrogen mixture.
1.3. Method 1.1 or 1.2, wherein the step is carried out using a flow reactor, e.g., a co-current flow reactor, optionally a falling film reactor, such as described in US 2017/0247306, the contents of which are hereby incorporated by reference in its entirety.
1.4. Method 1.1, 1.2, or 1.3, wherein the dihydromyrcenol is provided as a solution in a solvent (e.g., in an alcohol or carboxylic acid solvent), or as an aqueous emulsion (e.g., a water/alcohol or water/carboxylic acid emulsion), or wherein the dihydromyrcenol is neat (i.e., not dissolved in any solvent).
1.5. Method 1 or any of 1.1-1.5, wherein the method further comprises the step of treating the ozonide intermediate with a reducing agent (also known as a quenching agent).
1.6. Method 1.5, wherein the reducing agent is selected from hydrogen gas (e.g., with a metal catalyst, such as palladium, platinum, rhodium, etc.), sodium bisulfite, sodium metabisulfite, hydroxymethanesulfinic acid, sodium hydroxymethanesulfinate, dimethyl sulfide, thiourea, zinc metal, triphenylphosphine, borohydride salts, aluminum hydride salts, and formic acid.
1.7. Any of methods 1.1-1.6, wherein the solvent for the reaction (e.g., for the ozonation step and/or the reduction step) is selected from water, methanol, ethanol, propanol, isopropanol, n-butanol, s-butanol, t-butanol, acetic acid, propionic acid, butyric acid, formic acid, n-propyl acetate, isopropyl acetate, n-butyl acetate, t-butyl acetate, ethyl propionate, propyl propionate, butyl propionate, methyl butyrate, ethyl butyrate, hexane, cyclohexane, heptane, octane, or a mixture thereof, e.g., wherein the solvent is selected from water, isopropanol, n-butanol, acetic acid, propionic acid, butyric acid, formic acid, n-butyl acetate, cyclohexane, hexane, heptane, or a mixture thereof.
1.8. Any of methods 1.1-1.6, wherein the reaction (e.g., the ozonation step and/or the reduction step) is carried out neat, i.e., without added solvent.
1.9. Any of methods 1.1-1.8, wherein the reduction step is carried out with an aqueous solution of the reducing agent (e.g., sodium sulfite, sodium metabisulfite, hydroxymethanesulfinic acid, or sodium hydroxymethanesulfinate), optionally with a water-miscible cosolvent or buffer.
1.10. Method 1.9, wherein the cosolvent is a water-miscible organic solvent, such as methanol, ethanol, isopropanol, acetonitrile, DMSO, DMF, dioxane, or tetrahydrofuran.
1.11. Method 1.9 or 1.10, wherein the buffer is an inorganic basic salt such as an alkali metal acetate (e.g., sodium acetate, potassium acetate) or alkali metal phosphate (e.g., sodium phosphate, potassium phosphate), or alkali metal carbonate or bicarbonate (e.g., sodium carbonate, sodium bicarbonate, potassium carbonate, potassium bicarbonate).
1.12. Any of Methods 1.1-1.11, where the initial product recovered from the reaction is purified, e.g., by chromatography, distillation, and/or aqueous extraction, to provide a mixture of hydroxymelonal and hydroxymelonal formate.
1.13. Method 1.12, wherein an initial product mixture comprising or consisting of hydroxymelonal and hydroxymelonal formate is further purified, e.g., by distillation, to increase or decrease the proportion of hydroxymelonal formate, such as to isolate hydroxymelonal formate.
1.14. Method 1.12, wherein hydroxymelonal formate is isolated in pure or substantially pure form from the reaction products.
1.15. Method 1.12, wherein the final product obtained from the reaction (e.g., after purification) is a mixture of hydroxymelonal and hydroxymelonal formate in a mass ratio of 5:1 to 1000:1, e.g., 10:1 to 1000:1, or 20:1 to 1000:1, 25:1 to 1000:1, or 50:1 to 1000:1, or 75:1 to 1000:1, or 100:1 to 1000:1, or 200:1 to 1000:1, or 250:1 to 1000:1, or 500:1 to 1000:1 or 750:1 to 1000:1, or 5:1 to 500:1, or 10:1 to 500:1, or 20:1 to 500:1, 25:1 to 500:1, or 50:1 to 500:1, or 75:1 to 500:1, or 100:1 to 500:1, or 200:1 to 500:1, or 250:1 to 500:1, or 5:1 to 250:1, or 10:1 to 250:1, or 20:1 to 250:1, 25:1 to 250:1, or 50:1 to 250:1, or 75:1 to 250:1, or 100:1 to 250:1, or 200:1 to 250:1, or 5:1 to 100:1, or 10:1 to 100:1, or 20:1 to 100:1, 25:1 to 100:1, or 50:1 to 100:1, or 75:1 to 100:1, or 5:1 to 50:1, or 10:1 to 50:1, or 20:1 to 50:1, or 25:1 to 50:1, or 5:1 to 25:1, or 10:1 to 25:1, or 15:1 to 25:1, or 20:1 to 25:1, or 5:1 to 10:1, or 5:1 to 7.5:1, or about 5:1, or about 10:1, or about 15:1, or about 25:1, or about 30:1, or about 35:1, or about 40:1, or about 45:1 or about 50:1, or about 60:1, or about 70:1 or about 75:1, or about 85:1, or about 100:1, or about 125:1, or about 150:1, or about 200:1, or about 250:1, or about 500:1, or about 750:1, or about 1000:1.
1.16. Method 1.12, wherein the product obtained from the reaction (e.g., after purification) is a mixture of hydroxymelonal and hydroxymelonal formate in a molar ratio of 5:1 to 1000:1, e.g., 10:1 to 1000:1, or 20:1 to 1000:1, 25:1 to 1000:1, or 50:1 to 1000:1, or 75:1 to 1000:1, or 100:1 to 1000:1, or 200:1 to 1000:1, or 250:1 to 1000:1, or 500:1 to 1000:1 or 750:1 to 1000:1, or 5:1 to 500:1, or 10:1 to 500:1, or 20:1 to 500:1, 25:1 to 500:1, or 50:1 to 500:1, or 75:1 to 500:1, or 100:1 to 500:1, or 200:1 to 500:1, or 250:1 to 500:1, or 5:1 to 250:1, or 10:1 to 250:1, or 20:1 to 250:1, 25:1 to 250:1, or 50:1 to 250:1, or 75:1 to 250:1, or 100:1 to 250:1, or 200:1 to 250:1, or 5:1 to 100:1, or 10:1 to 100:1, or 20:1 to 100:1, 25:1 to 100:1, or 50:1 to 100:1, or 75:1 to 100:1, or 5:1 to 50:1, or 10:1 to 50:1, or 20:1 to 50:1, or 25:1 to 50:1, or 5:1 to 25:1, or 10:1 to 25:1, or 15:1 to 25:1, or 20:1 to 25:1, or 5:1 to 10:1, or 5:1 to 7.5:1, or about 5:1, or about 10:1, or about 15:1, or about 25:1, or about 30:1, or about 35:1, or about 40:1, or about 45:1 or about 50:1, or about 60:1, or about 70:1 or about 75:1, or about 85:1, or about 100:1, or about 125:1, or about 150:1, or about 200:1, or about 250:1, or about 500:1, or about 750:1, or about 1000:1
1.17. Method 1.12, wherein the product obtained from the reaction (e.g., after purification) is a mixture of hydroxymelonal and hydroxymelonal formate comprising or consisting of 0.1 to 20% by weight of hydroxymelonal formate and the balance being hydroxymelonal, e.g., wherein the composition consists of 0.1 to 15%, or 0.1 to 10%, or 0.1 to 7.5%, or 0.1 to 5%, or 0.1 to 4%, or 0.1 to 3%, or 0.1 to 2%, or 0.1 to 1%, or 0.1 to 0.8%, or 0.1 to 0.6%, or 0.1 to 0.5%, or 0.1 to 0.4%, or 0.1 to 0.3%, or 0.1 to 0.2%, or 0.1 to 0.15%, or 0.5% to 15%, or 0.5% to 10%, or 0.5 to 7.5%, or 0.5 to 5%, or 0.5 to 4%, or 0.5 to 3%, or 0.5 to 2%, or 0.5 to 1%, or 0.5 to 0.8%, or 0.5 to 0.6%, or 1 to 15%, or 1 to 10%, or 1 to 7.5%, or 1 to 5%, or 1 to 4%, or 1 to 3%, or 1 to 2%, or 1 to 1.5%, or 2 to 15%, or 2 to 10%, or 2 to 7.5%, or 2 to 5%, or 2 to 4%, or 2 to 3%, or 2 to 2.5%, or 3 to 15%, or 3 to 10%, or 3 to 7.5%, or 3 to 5%, or 5 to 15%, or 5 to 10%, or 5 to 7.5%, or 7.5% to 15%, or 7.5% to 10%, or 10 to 15%, by weight of hydroxymelonal formate and the balance being hydroxymelonal.
1.18. Any of Methods 1 or 1.1-1.17, wherein the method provides pure hydroxymelonal formate (e.g., >95%, >97%, >98%, or >99% pure) or a mixture consisting essentially of hydroxymelonal and hydroxymelonal formate (e.g., <5%, or <3%, or less than 2%, or less than 1%, of any other components).

Having now described some embodiments of the application, it should be apparent to those skilled in the art that the foregoing is merely illustrative and not limiting, having been presented by way of example only. The embodiments of the application can therefore be in other specific forms without departing from the spirit or essential characteristics thereof.

Those skilled in the art should recognize or be able to ascertain, using no more than routine experimentation, equivalents to the specific embodiments of the application. It is therefore to be understood that the embodiments described herein are presented by way of example only and that the scope of the application is thus indicated by the appended claims and equivalents thereto, and that the application may be practiced otherwise than as specifically described in the foregoing description.

The term “about,” when used to describe one of the compositions of the application, refers to a recited percentage±5%, ±4%, 3%, ±2.5%, ±2%, ±1.5%, ±1%, ±0.75%, ±0.5%, ±0.25%, or ±0.1%. In one embodiment, the term “about,” refers to a recited percentage±5%. For example, “about 50%” refers to the range 45% to 55%. In one embodiment, the term “about,” refers to a recited percentage±2.5%. In one embodiment, the term “about,” refers to a recited percentage±1%. In one embodiment, the term “about,” refers to a recited percentage±0.5%. In one embodiment, the term “about,” refers to a recited percentage±0.1%.

As used herein, the singular forms “a,” “an,” and “the” include plural referents unless the context clearly dictates otherwise. Thus, for example, reference to “a fragrance ingredient” includes not only a single fragrance ingredient but also a combination or mixture of two or more different fragrance ingredients, reference to “an additive” includes a single additive as well as two or more additives, and the like.

As used herein, the phrases “for example,” “for instance,” “such as,” or “including” are meant to introduce examples that further clarify more general subject matter. These examples are provided only as an aid for understanding the disclosure, and are not meant to be limiting in any fashion. Furthermore, as used herein, the terms “may,” “optional,” “optionally,” or “may optionally” mean that the subsequently described circumstance may or may not occur, so that the description includes instances where the circumstance occurs and instances where it does not. For example, the phrase “optionally present” means that an object may or may not be present, and, thus, the description includes instances wherein the object is present and instances wherein the object is not present.

The entire disclosure of each of the patent documents and scientific articles referred to herein is incorporated by reference in its entirety for all purposes. The function and advantages of these and other embodiments will be more fully understood from the following non-limiting examples. The examples are intended to be illustrative in nature and are not to be considered as limiting the scope of the embodiments discussed herein.

### EXAMPLES

### Example 1: Synthesis of Hydroxymelonal and Hydroxymelonal Formate

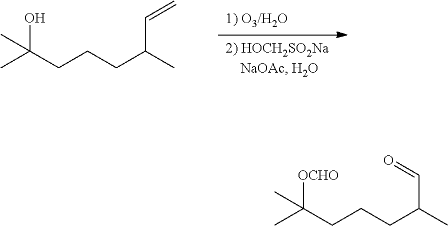

A well-mixed, heterogeneous mixture of dihydromyrcenol (40% wt/wt) and water (60% wt/wt) is pumped through two sequential pilot-plant scale falling film reactors at a flow rate of 1.9 kg/hour. In the reactors, the film is contacted with a co-current gas mixture of 3.7% w/w ozone in nitrogen, at a total gas flow of 136 standard liters per minute (slpm). The tube reactor jacket temperature is maintained at 5-10° C.

Continuous quenching of the output stream from the second reactor is performed using aqueous sodium acetate (25% wt/wt in water) at a 2% wt/wt loading as compared to the dihydromyrcenol, and sodium hydroxymethanesulfinate (Rongalite) (40% wt/wt in water), at 0.75 molar equivalent to the dihydromyrcenol, with mixing in a CSTR (continuous stirred tank reactor) throughout the continuous campaign under nitrogen atmosphere. The temperature of the CSTR is set to 65° C. with the actual temperature staying in the range of 65-68° C.

The reaction temperature is then reduced to 25° C., and then solid sodium chloride (in an amount to provide a 20% wt/wt solution) and tocopherol (0.15% wt/wt, as compared to the dihydromyrcenol loaded) are added into the vessel during the stirring. After all of the solids are dissolved, the aqueous phase is drained and discarded, and the organic layer is basified with 10% w/w aqueous sodium carbonate slowly until a pH of 10 is achieved. The separated aqueous phase is then drained and discarded. GC shows that all hydroxymelonoic acid (15.661 min peak in GC spectrum) has been removed. Following another phase separation, the organic layer is collected and carried onto to the distillation operation. Distillation is carried out to obtain the desired fractions. A mixture of hydroxymelonal and hydroxymelonal formate are together isolated. Further distillation may be performed to provide pure hydroxymelonal formate.

Similar results to the above are also obtained under experimental conditions utilizing hydrogen gas over a palladium catalyst for the reduction of the ozonide intermediates.

The Examples provided herein are exemplary only and are not intended to be limiting in any way to the various aspects and embodiments of the invention described herein.

1. The compound hydroxymelonal formate,

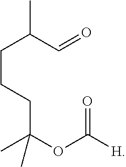

2. A flavor composition and/or fragrance composition comprising hydroxymelonal formate.

3. The composition of claim 2, wherein the composition further comprises one or more solvents.

4. The composition of claim 2, wherein the composition further comprises one or more other flavors or fragrances.

5. The composition of claim 2, wherein the composition further comprises hydroxymelonal.

6. The composition of claim 5, wherein the composition comprises hydroxymelonal and hydroxymelonal formate in a mass ratio of 5:1 to 1000:1, e.g., 10:1 to 1000:1, or 20:1 to 1000:1, 25:1 to 1000:1, or 50:1 to 1000:1, or 75:1 to 1000:1, or 100:1 to 1000:1, or 200:1 to 1000:1, or 250:1 to 1000:1, or 500:1 to 1000:1 or 750:1 to 1000:1, or 5:1 to 500:1, or 10:1 to 500:1, or 20:1 to 500:1, 25:1 to 500:1, or 50:1 to 500:1, or 75:1 to 500:1, or 100:1 to 500:1, or 200:1 to 500:1, or 250:1 to 500:1, or 5:1 to 250:1, or 10:1 to 250:1, or 20:1 to 250:1, 25:1 to 250:1, or 50:1 to 250:1, or 75:1 to 250:1, or 100:1 to 250:1, or 200:1 to 250:1, or 5:1 to 100:1, or 10:1 to 100:1, or 20:1 to 100:1, 25:1 to 100:1, or 50:1 to 100:1, or 75:1 to 100:1, or 5:1 to 50:1, or 10:1 to 50:1, or 20:1 to 50:1, or 25:1 to 50:1, or 5:1 to 25:1, or 10:1 to 25:1, or 15:1 to 25:1, or 20:1 to 25:1, or 5:1 to 10:1, or 5:1 to 7.5:1, or about 5:1, or about 10:1, or about 15:1, or about 25:1, or about 30:1, or about 35:1, or about 40:1, or about 45:1 or about 50:1, or about 60:1, or about 70:1 or about 75:1, or about 85:1, or about 100:1, or about 125:1, or about 150:1, or about 200:1, or about 250:1, or about 500:1, or about 750:1, or about 1000:1.

7. The composition of claim 5, wherein the composition comprises hydroxymelonal and hydroxymelonal formate in a molar ratio of 5:1 to 1000:1, e.g., 10:1 to 1000:1, or 20:1 to 1000:1, 25:1 to 1000:1, or 50:1 to 1000:1, or 75:1 to 1000:1, or 100:1 to 1000:1, or 200:1 to 1000:1, or 250:1 to 1000:1, or 500:1 to 1000:1 or 750:1 to 1000:1, or 5:1 to 500:1, or 10:1 to 500:1, or 20:1 to 500:1, 25:1 to 500:1, or 50:1 to 500:1, or 75:1 to 500:1, or 100:1 to 500:1, or 200:1 to 500:1, or 250:1 to 500:1, or 5:1 to 250:1, or 10:1 to 250:1, or 20:1 to 250:1, 25:1 to 250:1, or 50:1 to 250:1, or 75:1 to 250:1, or 100:1 to 250:1, or 200:1 to 250:1, or 5:1 to 100:1, or 10:1 to 100:1, or 20:1 to 100:1, 25:1 to 100:1, or 50:1 to 100:1, or 75:1 to 100:1, or 5:1 to 50:1, or 10:1 to 50:1, or 20:1 to 50:1, or 25:1 to 50:1, or 5:1 to 25:1, or 10:1 to 25:1, or 15:1 to 25:1, or 20:1 to 25:1, or 5:1 to 10:1, or 5:1 to 7.5:1, or about 5:1, or about 10:1, or about 15:1, or about 25:1, or about 30:1, or about 35:1, or about 40:1, or about 45:1 or about 50:1, or about 60:1, or about 70:1 or about 75:1, or about 85:1, or about 100:1, or about 125:1, or about 150:1, or about 200:1, or about 250:1, or about 500:1, or about 750:1, or about 1000:1.

8. The composition of claim 2, wherein the composition consists of hydroxymelonal and hydroxymelonal formate.

9. The composition of claim 8, wherein the composition consists of 0.1 to 20% by weight of hydroxymelonal formate, wherein the balance of the mass is hydroxymelonal, e.g., wherein the composition consists of 0.1 to 15%, or 0.1 to 10%, or 0.1 to 7.5%, or 0.1 to 5%, or 0.1 to 4%, or 0.1 to 3%, or 0.1 to 2%, or 0.1 to 1%, or 0.1 to 0.8%, or 0.1 to 0.6%, or 0.1 to 0.5%, or 0.1 to 0.4%, or 0.1 to 0.3%, or 0.1 to 0.2%, or 0.1 to 0.15%, or 0.5% to 15%, or 0.5% to 10%, or 0.5 to 7.5%, or 0.5 to 5%, or 0.5 to 4%, or 0.5 to 3%, or 0.5 to 2%, or 0.5 to 1%, or 0.5 to 0.8%, or 0.5 to 0.6%, or 1 to 15%, or 1 to 10%, or 1 to 7.5%, or 1 to 5%, or 1 to 4%, or 1 to 3%, or 1 to 2%, or 1 to 1.5%, or 2 to 15%, or 2 to 10%, or 2 to 7.5%, or 2 to 5%, or 2 to 4%, or 2 to 3%, or 2 to 2.5%, or 3 to 15%, or 3 to 10%, or 3 to 7.5%, or 3 to 5%, or 5 to 15%, or 5 to 10%, or 5 to 7.5%, or 7.5% to 15%, or 7.5% to 10%, or 10 to 15%, by weight of hydroxymelonal formate, and the balance of the mass is hydroxymelonal.

10. The composition of claim 2, wherein the composition comprises or consists of hydroxymelonal, hydroxymelonal formate, and one or more solvents.

11. The composition of claim 10, wherein the composition comprises or consists of hydroxymelonal, hydroxymelonal formate, and a net amount of solvent of 1 to 99% by weight of the composition, e.g., 10 to 99%, or 15 to 99%, or 20 to 99%, or 25 to 99%, or 30 to 99%, or 35 to 99%, or 40 to 99%, or 45 to 99%, or 50 to 99%, or 60 to 99%, or 70 to 99%, or 80 to 99%, or 90 to 99%, or 95 to 99%, or 98 to 99%, the balance being hydroxymelonal and hydroxymelonal formate.

12. The composition of claim 3, wherein the one or more solvents are selected from water, methanol, ethanol, propanol, isopropanol, dimethyl ether, diethyl ether, diisopropyl ether, methyl tert-butyl ether, ethylene glycol, propylene glycol, glycerin, triethyl citrate, triacetin, triglycerides, liquid waxes, propylene glycol derivatives, ethylene glycol derivatives, other alcohols or ethers, or any combination thereof.

13. The composition of claim 2, wherein the composition is a liquid.

14. The composition of claim 2, wherein the composition is a soft or waxy solid.

15. The composition of claim 2, wherein the composition further comprises one or more of a polymer, gelling agent, powdery substrate, surfactant, emollient, plasticizer, wetting agent, swelling agent, or active agent (e.g., an oral care active or a medicinal active agent), or any other additives as described herein.

16. A product comprising the composition of claim 2, wherein the product is selected from the following: personal care products (e.g., a soap, skin cream or lotion, balm, shampoo, body wash, shower gel, hydrating cream, deodorant, antiperspirant, after-shave lotion, cologne, perfume, or other hair care or skin care product), sunscreens, insect repellants and insecticides, detergents, household cleaning agents (e.g., a surface cleaner, a metal cleaner, a wood cleaner, a glass cleaner, a body cleaner such as a soap, a dish-washing detergent, or a laundry detergent), air fresheners, room sprays, pomanders, candles, cosmetics (e.g., perfumes, colognes, nail polish, eye liner, mascara, lipstick, foundation, concealer, blush, bronzer, eye shadow, lip liner, lip balm), toilet waters, talcum powders, and pet litter.

17. A method for the preparation of hydroxymelonal formate and/or hydroxymelonal, wherein the method comprises the step of reacting dihydromyrcenol with ozone for a sufficient amount of time to form an ozonide intermediate and/or to form the hydroxymelonal formate product by intramolecular reaction.

18. The method of claim 17, wherein the dihydromyrcenol is provided as a solution in a solvent (e.g., in an alcohol or carboxylic acid solvent), or as an aqueous emulsion (e.g., a water/alcohol or water/carboxylic acid emulsion), or wherein the dihydromyrcenol is neat (i.e., not dissolved in any solvent).

19. The method of claim 17, wherein the method further comprises the step of treating the ozonide intermediate with a reducing agent, optionally wherein the reducing agent is selected from hydrogen gas (e.g., with a metal catalyst, such as palladium, platinum, rhodium, etc.), sodium bisulfite, sodium metabisulfite, hydroxymethanesulfinic acid, sodium hydroxymethanesulfinate, dimethyl sulfide, thiourea, zinc metal, triphenylphosphine, borohydride salts, aluminum hydride salts, and formic acid.

20. The method of claim 17, wherein the method provides pure hydroxymelonal formate (e.g., >95%, >97%, >98%, or >99% pure) or a mixture consisting essentially of hydroxymelonal and hydroxymelonal formate (e.g., <5%, or <3%, or less than 2%, or less than 1%, of any other components).
